(19) United States
(12) Patent Application Publication
(10) Pub. No.: US 2019/0179831 A1
(43) Pub. Date: Jun. 13, 2019

(54) SYSTEMS, METHODS, AND PORTABLE DEVICES FOR UPDATING USER INFORMATION

(71) Applicant: Visionary Technologies LLC, St. Charles, MO (US)

(72) Inventors: Ronald W. ZAMBER, Fairbanks, AK (US); Allen A.S. ALI, Wilmette, IL (US); Patrick RILEY, Calabasas, CA (US); Leonard NELSON, Raleigh, NC (US)

(21) Appl. No.: 16/215,443

(22) Filed: Dec. 10, 2018

Related U.S. Application Data
(60) Provisional application No. 62/596,740, filed on Dec. 8, 2017.

(51) Int. Cl. G06F 16/27; G06F 16/29; G06F 16/23; G16H 10/60; H04W 4/029

(52) U.S. Cl. CPC G06F 16/275; G06F 16/29; H04W 4/029; G16H 10/60; G06F 16/2379

(57) ABSTRACT

A system and method for updating user information includes a first device with first circuitry. The first circuitry is configured to generate motion data of the first device, and receive time-stamped location data of the first device from at least one communication device based on the generated motion data. The first circuitry is further configured to generate time synchronized user data based on the time-stamped location data and device data of the first device, and transmit the time synchronized user data. The system further comprises a second device communicably coupled to the first device. The second device comprises second circuitry configured to receive the time synchronized user data and update the user information based on the time synchronized user data.

### FIELD OF THE PRESENT DISCLOSURE

An example embodiment of the present invention generally relates to indoor positioning systems, and more particularly relates to a system, method, and portable device for updating user information.

### BACKGROUND

In a multi-storied building having many rooms and corridors, it may be difficult to locate and find a person. In present days, even though everybody may carry a smartphone, it may be difficult to communicate with the person in an urgent situation. For example, while walking in a big building, an elderly person may suddenly suffer a medical emergency (e.g. stroke). It may be time consuming to locate the elderly person and take him/her for treatment. Considering the time factor taken for locating him/her, it may even turn fatal. In such cases, continuous monitoring of people and recording information about their movements in some central location may mitigate mishaps. In conventional healthcare facilities, doctors write medical information, for example, diagnosis and prescribed drugs in a medical record. Keeping track of prescription, patient information, test reports, schedules etc. is difficult and cumbersome for patients as well as doctors. Also, present wearable bands or smart watches may only transmit health parameters to remote computers, where the doctors may remotely address the patients. Accordingly, there is a need for a more efficient and cost-effective system for updating user information.

### SUMMARY

A system, method, and portable device are provided in accordance with an example embodiment described herein for updating user information.

In one aspect, a system for updating user information is disclosed. The system comprises a first device comprising first circuitry and a second device comprising second circuitry, communicably coupled to the first device. The first circuitry is configured to generate motion data of the first device; receive time-stamped location data of the first device from at least one communication device, based on the generated motion data; generate time synchronized user data based on the time-stamped location data and device data of the first device; and transmit the time synchronized user data. The second circuitry is configured to receive the time synchronized user data; and update the user information based on the time synchronized user data.

In another aspect, a portable device is disclosed. The portable device comprises circuitry configured to generate motion data of the portable device; receive time-stamped location data of the portable device from at least one communication device, based on the generated motion data; generate time synchronized user data based on the time-stamped location data and device data of the portable device; and transmit the time synchronized user data to a server device for update of user information.

In yet another aspect, a method for updating user information is disclosed. The method comprises generating by a first device, motion data of the first device; receiving by the first device, time-stamped location data of the first device from at least one communication device based on the generated motion data; generating by the first device, time synchronized user data, based on the time-stamped location data and device data of the first device; and updating by a second device, the user information based on the time synchronized user data.

According to some embodiments, the time-stamped location data comprises geolocation data of the first device and timestamp data associated with the geolocation data.

According to some embodiments, the device data comprises device identifier data of the first device and profile data of a user associated with the first user device.

According to some embodiments, the profile data of the user comprises one or more of name, age, sex, health insurance number, address, medical history of the user.

According to some embodiments, the second circuitry is further configured to store the user information, wherein the user information corresponds to an electronic medical record of a user of the first device.

According to some embodiments, the time synchronized user data comprises geolocation data of the first device, timestamp data associated with the geolocation data, device identifier data of the first device, and profile data of a user of the first device.

According to some embodiments, the first device is a wearable device.

According to some embodiments, the communication device is an RFID transponder.

According to some embodiments, the second device is a database server.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

Having thus described example embodiments of the invention in general terms, reference will now be made to the accompanying drawings, which are not necessarily drawn to scale, and wherein:

FIG. 1 illustrates an exemplary scenario of updating user information, in accordance with one or more example embodiments;

FIG. 2 illustrates a schematic block diagram of a user information updating system, in accordance with one or more example embodiments;

FIG. 3 illustrates a flowchart depicting steps in a method for updating user information, in accordance with one or more example embodiments;

FIG. 4 illustrates a block diagram view of a portable device, in accordance with one or more exemplary embodiments;

FIG. 5 illustrates a flowchart depicting workflow of the portable device, in accordance with one or more example embodiments;

FIGS. 6A and 6B illustrate diagrammatic views of time-stamped location data generation, in accordance with one or more example embodiments;

FIG. 7 illustrates a schematic block diagram of a communication diagram, in accordance with one or more example embodiments;

FIG. 8 illustrates block diagram view of the electronic device, in accordance with one or more example embodiments;

FIG. 9 illustrates a flowchart depicting steps in a method for controlling transmission of contextual data, in accordance with one or more example embodiments; and

FIG. 10 is a diagram of a mobile terminal (e.g., handset or part thereof) that may be used to implement an embodiment.

### DETAILED DESCRIPTION

In the following description, for purposes of explanation, numerous specific details are set forth in order to provide a thorough understanding of the present disclosure. It will be apparent, however, to one skilled in the art that the present disclosure can be practiced without these specific details. In other instances, apparatuses and methods are shown in block diagram form only in order to avoid obscuring the present disclosure.

Reference in this specification to “one embodiment” or “an embodiment” means that a particular feature, structure, or characteristic described in connection with the embodiment is included in at least one embodiment of the present disclosure. The appearance of the phrase “in one embodiment” in various places in the specification are not necessarily all referring to the same embodiment, nor are separate or alternative embodiments mutually exclusive of other embodiments. Further, the terms “a” and “an” herein do not denote a limitation of quantity, but rather denote the presence of at least one of the referenced items. Moreover, various features are described which may be exhibited by some embodiments and not by others. Similarly, various requirements are described which may be requirements for some embodiments but not for other embodiments.

Some embodiments of the present invention will now be described more fully hereinafter with reference to the accompanying drawings, in which some, but not all, embodiments of the invention are shown. Indeed, various embodiments of the invention may be embodied in many different forms and should not be construed as limited to the embodiments set forth herein; rather, these embodiments are provided so that this disclosure will satisfy applicable legal requirements. Like reference numerals refer to like elements throughout. As used herein, the terms “data,” “content,” “information,” and similar terms may be used interchangeably to refer to data capable of being transmitted, received and/or stored in accordance with embodiments of the present invention. Thus, use of any such terms should not be taken to limit the spirit and scope of embodiments of the present invention.

Additionally, as used herein, the term ‘circuitry’ may refer to (a) hardware-only circuit implementations (for example, implementations in analog circuitry and/or digital circuitry); (b) combinations of circuits and computer program product(s) comprising software and/or firmware instructions stored on one or more computer readable memories that work together to cause an apparatus to perform one or more functions described herein; and (c) circuits, such as, for example, a microprocessor(s) or a portion of a microprocessor(s), that require software or firmware for operation even if the software or firmware is not physically present. This definition of ‘circuitry’ applies to all uses of this term herein, including in any claims. As a further example, as used herein, the term ‘circuitry’ also includes an implementation comprising one or more processors and/or portion(s) thereof and accompanying software and/or firmware. As another example, the term ‘circuitry’ as used herein also includes, for example, a baseband integrated circuit or applications processor integrated circuit for a mobile phone or a similar integrated circuit in a server, a cellular network device, other network device, and/or other computing device.

The embodiments are described herein for illustrative purposes and are subject to many variations. It is understood that various omissions and substitutions of equivalents are contemplated as circumstances may suggest or render expedient but are intended to cover the application or implementation without departing from the spirit or the scope of the present disclosure. Further, it is to be understood that the phraseology and terminology employed herein are for the purpose of the description and should not be regarded as limiting. Any heading utilized within this description is for convenience only and has no legal or limiting effect.

### Definitions

The term “portable device” may be used to refer to any user accessible device such as a mobile phone, a smartphone, a portable computer, and the like that is portable in itself or as a part of another portable object.

The term “polling” may be used to refer to a communication technique by which a computer or an electronic device checks if any peripheral devices are ready to connect to the computer or the electronic device

The term ‘device identifier data’ may be used to refer to data associated with a device to identify the device.

The term ‘unique identifier’ may be used to refer to unique signal transmitted by a device, which signifies identity and authenticity of the device.

The term ‘pre-selected distance’ may be used to refer to a region around a device, in which the device may connect with one or more other device/systems.

### End of Definitions

A system, method and portable device are provided herein in accordance with an example embodiment for updating user information. In some example embodiments, the system, method and portable device provided herein may also be used for locating a user within premises of an organization or a facility.

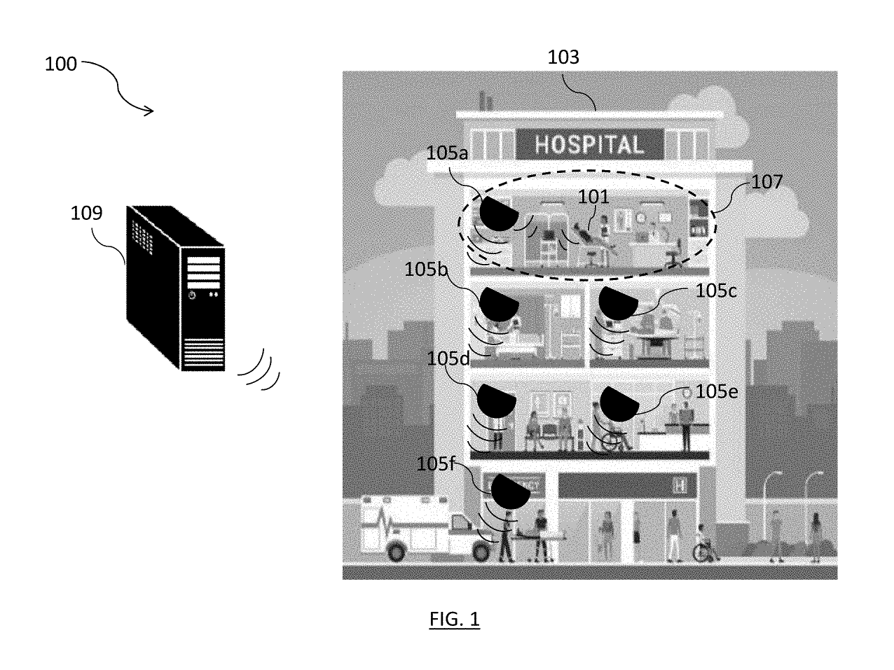

FIG. 1 illustrates an exemplary scenario of updating user information, in accordance with one or more example embodiments. In the exemplary scenario 100 of FIG. 1, a user carrying a first user device 101 may be present in Ear, Nose and Throat (ENT) department of a healthcare facility, for example, a hospital 103. In the exemplary scenario 100 of FIG. 1, a user information updating system may be implemented in the hospital 103 only as an example of the many applications of the user information updating system, including any facility or organization with people who moved within and outside of it.

The user may be a patient and the like who may be stationary or in motion with respect to the hospital 103. The first user device 101 may communicate with an electronic device 109 to transmit required information. There may be a plurality of communication devices (105c-105f), each may be configured to continuously transmit a unique identifier for polling. In some example embodiments, the plurality of the communication devices (105c-105f) may include radio frequency identification (RFID) transponders. The plurality of communication devices (105c-105f) may be configured for continuous polling to detect if the first user device 101 is within a pre-selected distance (PSD) corresponding to each communication device. The pre-selected distance (PSD) may be determined by an administrator of the user information updating system or may be set automatically by the user information updating system itself. Each of the plurality of communication devices (105c-105f) may also be configured to initiate a communication with the first user device 101 when the first user device 101 is within the pre-selected distance (PSD) corresponding to each communication device. For example, as shown in scenario 100 of FIG. 1, the first user device 101 may communicate with a communication device (e.g. communication device 105a) placed in the ENT department of the hospital 103. The communication device 105a may check if the first user device 101 is within a pre-selected distance (PSD) 107 corresponding to the communication device 105a, and may establish a communication with the first user device 101 when the first user device 101 comes within the pre-selected distance (PSD) 107. In some example embodiments, the plurality of communication devices (105c-105f) may be placed in different sections or areas of the hospital 103 and each of the plurality of communication devices (105c-105f) may correspond to a particular section or area of the hospital 103, where. it is placed. In some example embodiments, different sections or areas may include billing counter, emergency ward, medicine window and the like.

In some example embodiments, a controller may control the plurality of communication devices (105c-105f), the controller may define the coordinates of the particular section or area associated with each of the plurality of communication devices (105c-105f). The controller may also issue the unique identifier to each of the plurality of communication devices (105c-105f), and control the continuous polling of the plurality of communication devices (105c-105f). The first user device 101 may determine identity and authenticity of each of the plurality of communication devices (105c-105f) based on the unique identifier.

In some example embodiments, each of the plurality of communication devices (105c-105f) may include a processor, a non-transitory memory and a communication interface. Additional, fewer, or different components may also be possible. In one or more example embodiments, the communication interface may be one or more transceivers. The plurality of communication devices (105c-105f) may connect with the controller via the communication interface through wired or wireless communication network or any combination of wired and wireless communication networks including Wi-Fi, ZIGBEE, SCADA, Bluetooth, NFC and the like. The processor may be configured to receive the unique identifier corresponding to each of the plurality of communication devices (105c-105f) from the controller and encrypt the same. The non-transitory memory may be configured to store the encrypted unique identifier. The processor of each of the plurality of communication devices (105c-105f) may be further configured to detect presence of the first user device 101 when it is within the pre-selected distance (PSD). (e.g. processor of the communication device 105a may detect presence of the first user device 101 when the first user device 101 comes within the PSD 107). The processor may send time-stamped location data and the encrypted unique identifier (e.g. unique identifier corresponding to communication device 105a) to the first user device 101 via the communication interface. The processor may transmit the encrypted unique identifier separately or with the time-stamped location data.

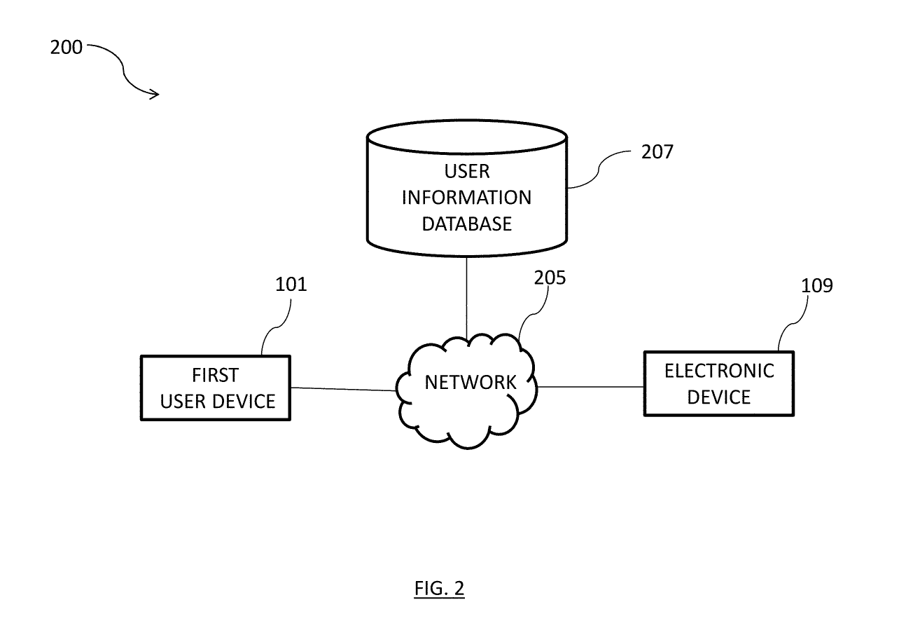

FIG. 2 illustrates a schematic block diagram of a user information updating system, in accordance with one or more example embodiments. The user information updating system of FIG. 2 may comprise the first user device 101, the electronic device 109 and a user information database 207. As shown in the block diagram 200 of FIG. 2, the first user device 101 may be communicatively coupled to the electronic device 109 via a communication network 205. The first user device 101 may comprise first circuitry configured to perform different functionalities. The electronic device may comprise second circuitry to carry out functionalities required for working of the user information updating system. The terms “user information updating system” and “system” may be used to refer to the user information updating system depicted in the block diagram 200 of FIG. 2, and may be used interchangeably hereinafter. The system may comprise one or more physical packages (for example, chips) including materials, components and/or wires on a structural assembly (for example, a baseboard). The structural assembly may provide physical strength, conservation of size, and/or limitation of electrical interaction for component circuitry included thereon.

In some example embodiments, the first user device 101 may be a portable device, such as a healthcare-wrist band, a health and wellness card, a smart watch and the like. The first user device 101 may be configured to be wearable or handheld. The first user device 101 may contain device data, utilized by the system of FIG. 2 to identify the first user device 101. In one or more example embodiments, the electronic device 209 may be a database server. In one or more alternative or additional embodiments, the electronic device 209 may also be a wireless device, same as the first user device 101, daisy-chained with the first user device 101 for purposes of communicating information.

In some example embodiments, the communication network 205 may be wired, wireless, or any combination of wired and wireless communication networks. The wired network may include a Local Area Network (LAN), a Metro Area Network (MAN), a Wide Area Network (WAN) and the like. Further, the wireless network may include satellite network such as cellular (for example, 4G, 3G, CDMA, WCDMA, and the like.), Wi-Fi, internet, or the like. The user information database 207. may store information about the user, such as name, age, sex, health insurance number, address, medical history, and the like.

The user information database 207 may be a master database stored in a format that facilitates updating, maintenance, and development. For example, data in the master database may be in an Oracle spatial format or other spatial format, such as for development or production purposes. The Oracle spatial format or development/production database may be compiled into a delivery format, such as a geographic data files (GDF) format. The data in the production and/or delivery formats may be compiled or further compiled to form user database products or databases, which may be used in end user information updating devices or systems.

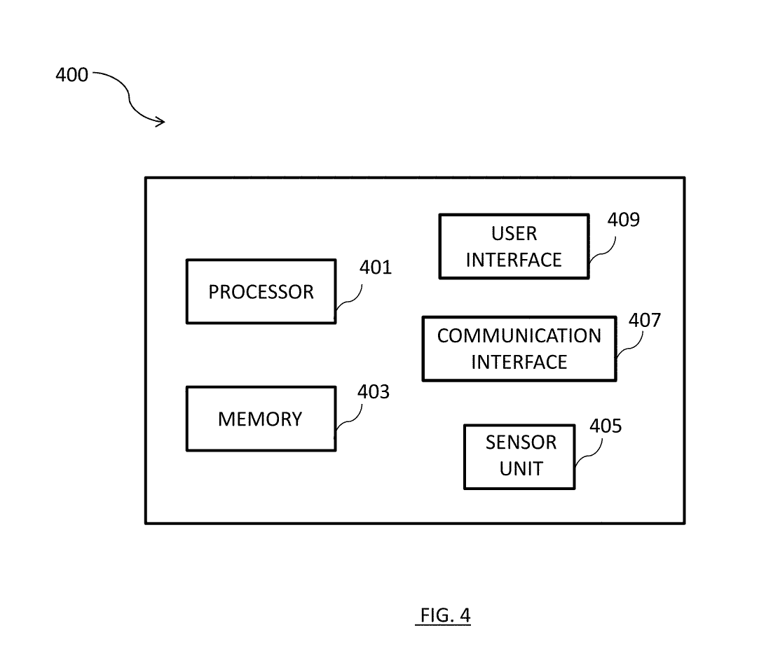

The first circuitry may comprise at least one processor, at least one memory, a communication interface and a sensor unit. Additional, fewer, or different components may also be possible. The processor may be embodied in a number of different ways, similar to a processor 401 as exemplarily illustrated in FIG. 4. For example, the processor of the first user device 101 may be embodied as one or more of various hardware processing means such as a coprocessor, a microprocessor, a controller, a digital signal processor (DSP), a processing element with or without an accompanying DSP, or various other processing circuitry including integrated circuits such as, for example, an ASIC (application specific integrated circuit), an FPGA (field programmable gate array), a microcontroller unit (MCU), a hardware accelerator, a special-purpose computer chip, or the like. The memory of the first user device 101 may be non-transitory and may include, for example, one or more volatile and/or non-volatile memories, similar to a memory 403 as exemplarily illustrated in FIG. 4. For example, the memory may be an electronic storage device (for example, a computer readable storage medium) comprising gates configured to store data (for example, bits) that may be retrievable by a machine (for example, a computing device like the processor). The communication interface of the first user device 101 may comprise input interface and output interface for supporting communications to and from the first user device 101. The communication interface may be any means such as a device or circuitry embodied in either hardware or a combination of hardware and software that is configured to receive and/or transmit data to/from a communications device in communication with the first user device 101, similar to a communication interface 407 as exemplarily illustrated in FIG. 4. The sensor unit of the first user device 101 may comprise one or more sensors, similar to a sensor unit 405 as exemplarily illustrated in FIG. 4. The one or more sensors may be configured to find location of the first user device, to detect movements of the first user device 101 when the first user device is moved from one location to another and the like.

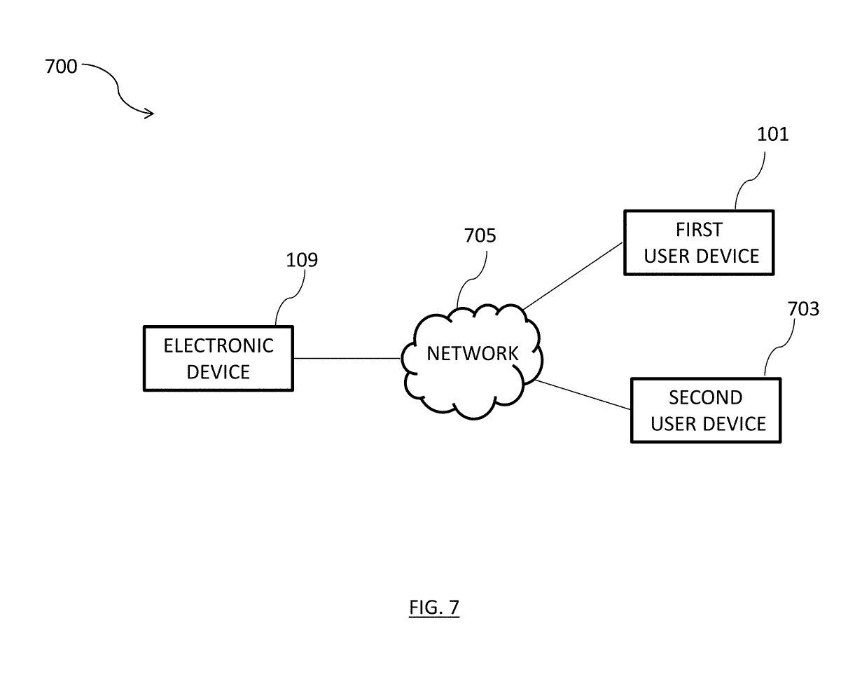

The second circuitry may comprise processing means and communication means. For example, the processing means may comprise one or more processors configured to process requests received from the first user device 101. The processing means may fetch user information from the user information database 207 and transmit the same to the first user device 101 in a format suitable for use by the first user device 101. In some alternative embodiments, the second circuitry may be configured to store user information. Accordingly, further in some another example embodiments, the user information may also be stored in the first user device 101. In some example embodiments, the electronics device 109 may control transmission of contextual data from the first user device 101 to one or more devices/systems, as described in communication diagram 700 of FIG. 7. The contextual data may include one or more of personal data of the user of the first user device 101 or the user information.

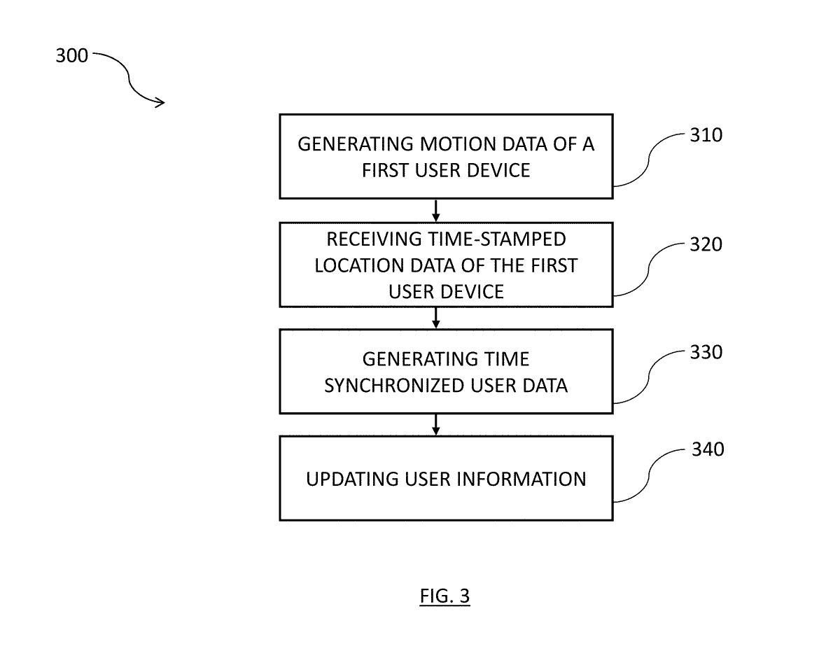

FIG. 3 illustrates a flowchart illustrative of a method according to example embodiments of the present invention. Accordingly, blocks of the flowcharts support combinations of means for performing the specified functions and combinations of operations for performing the specified functions for performing the specified functions. It will also be understood that one or more blocks of the flowcharts, and combinations of blocks in the flowcharts, can be implemented by special purpose hardware-based computer systems which perform the specified functions, or combinations of special purpose hardware and computer instructions.

FIG. 3 illustrates a flowchart depicting steps in a method 300 for updating user information, according to an example embodiment of the present invention. The method 300 may comprise at 310, generating motion data of the first user device 101. The method 300 may comprise, at 320, receiving time-stamped location data of the first user device 101 based on the generated motion data, from at least one communication device (e.g. the communication device 105a). The time-stamped location data may comprise geolocation data of the first user device 101 and timestamp data associated with the geolocation data. The method 300 may further comprise, at 330, generating by the first user device 101, time synchronized user data, based on the time-stamped location data and device data of the first user device 101. The time synchronized user data may comprise geolocation data of the first user device 101, timestamp data associated with the geolocation data, device identifier data of the first user device 101, and profile data of the user of the first user device 101. The device data may comprise device identifier data of the first user device 101 and profile data of a user associated with the first user device 101. The profile data of the user may comprise one or more of name, age, sex, health insurance number, address, medical history of the user. The method 300 may further comprise, at 340, updating by the electronic device 109, the user information based on the time synchronized user data. At this step, the electronic device 109 may receive the time synchronized user data from the first user device 101 which may be communicably coupled to the electronic device. The user information may correspond to an electronic medical record of the user associated with the first user device 101. Hereinafter, the terms “device identifier data” and “first user device identification data” may be interchangeably used to refer to device identifier data of the first user device 101.

Additionally, various other steps not shown in FIG. 3 may also be included in the method 300. For example, the method 300 may further comprise receiving a unique identifier corresponding to the at least one communication device (e.g. the communication device 105a). The method 300 may further comprise sending device data of the first user device 101 to the at least one communication device (e.g. the communication device 105a). The first user device 101 may authenticate the at least one communication device (e.g. the communication device 105a) based on the received unique identifier. Likewise, the device data may be used by the at least one communication device (e.g. the communication device 105a). for identifying the first user device 101

In an example embodiment, a system (e.g., the system of FIG. 2) for performing the method of FIG. 3 above may comprise a processor configured to perform some or each of the operations (310-340) described above. The processor may, for example, be configured to perform the operations by performing hardware implemented logical functions, executing stored instructions, or executing algorithms for performing each of the operations. Alternatively, the system may comprise means for performing each of the operations described above. In this regard, according to an example embodiment, examples of means for performing operations 310-340 may comprise, for example, the processor and/or a device or circuit for executing instructions or executing an algorithm for processing information as described above.

Implementation of example embodiments disclosed herein may be explained, with reference to the exemplary scenario 100 of FIG. 1, as follows. When the user reaches the ENT department of the hospital 103 and the first user device 101 comes within PSD 107 of the communication device 105a placed in the ENT department, the communication device 105a may communicate with the first user device 101. On building connection with the first user device 101, the communication device 105a may share its unique identifier with the first user device 101. The sensor unit of the first user device 101 may detect movement of the first user device 101 (i.e. movement of the user). On detection of movement of the user, the processor of the first user device 101 may actuate the communication interface of the first user device 101 and may receive unique identifier of the communication device 105a via the communication interface. The processor of the first user device 101 may further send device data of the first user device 101 to the communication device 105a. On exchange of the unique identifier and the device data between the first user device 101 and the communication device 105a, the first user device 101 may receive time stamped location data of the first user device. The processor of the first user device 101 may generate time synchronized user data based on the time-stamped location data and device data of the first user device 101. The processor of the first user device 101 may further transmit the time synchronized user data to the electronic device 109. On receiving the time synchronized user data, processing means of the electronic device 109 may update the user information based on the time synchronized user data.

FIG. 4 illustrates a block diagram view of a portable device 400, in accordance with one or more exemplary embodiments. The portable device 400 may comprise circuitry required for specific functionalities of the portable device 400. The circuitry may comprise at least one processor 401, at least one memory 403, a sensor unit 405, a communication interface 407, and a user interface 409. Additional, fewer, or different components may also be possible.

In some example embodiments, the portable device 400 may be embodied as a chip or chip set. In other words, the portable device 400 may comprise one or more physical packages (for example, chips) including materials, components and/or wires on a structural assembly (for example, a baseboard). The structural assembly may provide physical strength, conservation of size, and/or limitation of electrical interaction for component circuitry included thereon. The portable device 400 may therefore, in some cases, be configured to implement an example embodiment of the present invention on a single “system on a chip.” As such, in some cases, a chip or chipset may constitute a means for performing one or more operations for providing the functionalities described herein.

The at least one processor 401 may be embodied in a number of different ways. For example, the processor may be embodied as one or more of various hardware processing means such as a coprocessor, a microprocessor, a controller, a digital signal processor (DSP), a processing element with or without an accompanying DSP, or various other processing circuitry including integrated circuits such as, for example, an ASIC (application specific integrated circuit), an FPGA (field programmable gate array), a microcontroller unit (MCU), a hardware accelerator, a special-purpose computer chip, or the like. As such, in some embodiments, the processor may include one or more processing cores configured to perform independently. A multi-core processor may enable multiprocessing within a single physical package. Additionally, or alternatively, the processor may include one or more processors configured in tandem via the bus to enable independent execution of instructions, pipelining and/or multithreading.

The at least one memory 403 may be non-transitory and may include, for example, one or more volatile and/or non-volatile memories. For example, the memory 403 may be an electronic storage device (for example, a computer readable storage medium) comprising gates configured to store data (for example, bits) that may be retrievable by a machine (for example, a computing device like the processor). The memory 403 may be configured to store information, data, content, applications, instructions, or the like, for enabling the apparatus to carry out various functions in accordance with an example embodiment of the present invention. For example, the memory 403 could be configured to buffer input data for processing by the processor. Additionally, or alternatively, the memory 403 could be configured to store instructions for execution by the processor.

The processor 401 (and/or co-processors or any other processing circuitry assisting or otherwise associated with the processor 401) may be in communication with the memory 403 via a bus for passing information among components of the portable device 400. The processor 401 may be configured to execute instructions stored in the memory 403 or otherwise accessible to the processor 401. Additionally, or alternatively, the processor 401 may be configured to execute hard coded functionality. As such, whether configured by hardware or software methods, or by a combination thereof, the processor 401 may represent an entity (for example, physically embodied in circuitry) capable of performing operations according to an embodiment of the present invention while configured accordingly. Thus, for example, when the processor 401 is embodied as an ASIC, FPGA or the like, the processor 401 may be specifically configured hardware for conducting the operations described herein. Alternatively, as another example, when the processor 401 is embodied as an executor of software instructions, the instructions may specifically configure the processor 401 to perform the algorithms and/or operations described herein when the instructions are executed. The processor 401 may include, among other things, a clock, an arithmetic logic unit (ALU) and logic gates configured to support operation of the portable device 400.

The sensor unit 405 may comprise one or more sensors. The one or more sensors may be configured to capture data of the portable device 400. In some example embodiments, the one or more sensors may include position sensors such as the GNSS sensor, e.g. GPS, Galileo, GLONASS, BeiDou signals or the like, motion sensor such as accelerometer, magnetic field sensors such as a magnetometer and/or compass, orientation sensor such as a gyroscope, luminosity sensor, image sensor such as a camera and the like. In some example embodiments, the one or more sensors may capture data of the portable device 400 such as but not limited to motion of the portable device 400, speed, acceleration, location, heading direction of the portable device 400 and the like. In some example embodiments, the one or more sensors may capture the data in real-time.

The communication interface 407 may comprise input interface and output interface for supporting communications to and from the portable device 400. The communication interface 407 may be any means such as a device or circuitry embodied in either hardware or a combination of hardware and software that is configured to receive and/or transmit data to/from a communications device in communication with the portable device 400. In this regard, the communication interface 407 may include, for example, an antenna (or multiple antennae) and supporting hardware and/or software for enabling communications with a wireless communication network. Additionally or alternatively, the communication interface 407 may include the circuitry for interacting with the antenna(s) to cause transmission of signals via the antenna(s) or to handle receipt of signals received via the antenna(s). In some environments, the communication interface 407 may alternatively or additionally support wired communication. As such, for example, the communication interface 407 may include a communication modem and/or other hardware and/or software for supporting communication via cable, digital subscriber line (DSL), universal serial bus (USB) or other mechanisms.

The user interface 409 may provide output to the user and, in some embodiments, to receive an indication of a user input. As such, the user interface 409 may include a display and, in some embodiments, may also include a keyboard, a mouse, a joystick, a touch screen, touch areas, soft keys, one or more microphones, a plurality of speakers, or other input/output mechanisms. The display may include a Liquid Crystal Display (LCD), a Light Emitting Diode display (LED), an Organic LED, and Amorphous LED, or a plasma screen. The display may be configured to display summarized or detailed information. In one embodiment, the processor 401 may comprise user interface circuitry configured to control at least some functions of one or more user interface elements such as a display and, in some embodiments, a plurality of speakers, a ringer, s and/or the like. The processor 401 and/or user interface circuitry comprising the processor 401 may be configured to control one or more functions of one or more user interface elements through computer program instructions (for example, software and/or firmware) stored on the memory 403 accessible to the processor 401. In some example embodiments, the user interface 409 may be embodied as a touch screen display. The user interface 409 may comprise one of a resistive type or a capacitive type touch surface as the user interface 409.

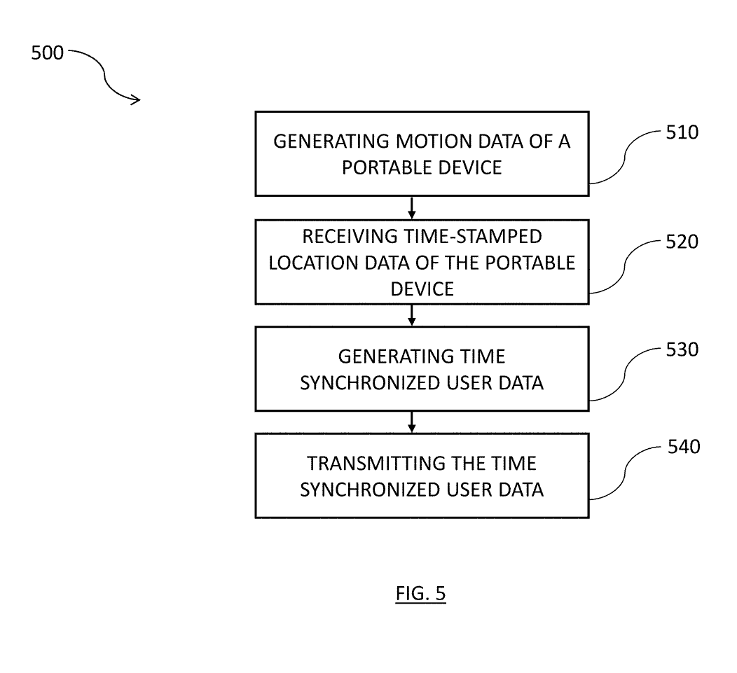

FIG. 5 illustrates a flowchart illustrative of a workflow, according to example embodiments of the present invention. Accordingly, blocks of the flowcharts support combinations of means for performing the specified functions and combinations of operations for performing the specified functions for performing the specified functions. It will also be understood that one or more blocks of the flowcharts, and combinations of blocks in the flowcharts, can be implemented by special purpose hardware-based computer systems which perform the specified functions, or combinations of special purpose hardware and computer instructions.

FIG. 5 illustrates a flowchart depicting workflow 500 of the portable device 400, in accordance with one or more example embodiments. At step 510, the sensor unit 411 may detect movement of the portable device 400 and may generate motion data. On receiving motion data from the sensor unit 411, the processor 401 may enable the communication interface 407 for data exchange with one or more external devices or systems. The memory 403 may store received/generated information. At step 520, the processor 401 may receive time stamped location data of the portable device 400 from at least one communication device, through the communication interface 407 based on the generated motion data. At step 530, the processor 401 may generate time synchronized user data based on the time-stamped location data and device data of the portable device 400. The memory 403 may further store device data of the portable device 400. At step 540, the processor 401 may transmit the time synchronized user data to a server device, through the communication interface 407, for update of user information. In some additional example embodiments, the processor 401 may download updated user information and may store the same in the memory 403. The user interface 409 may enable a user of the portable device 400 to view the updated user information.

Additionally, various other steps not shown in FIG. 5 may also be included in the workflow 500. For example, the processor 401 may receive a unique identifier corresponding to the at least one communication device through the communication interface 407. The processor 401 may send device data of the portable device 400 to the at least one communication device. The processor 401 may authenticate the at least one communication device based on the received unique identifier. Likewise, the device data may be used by the at least one communication device for identifying the portable device 400.

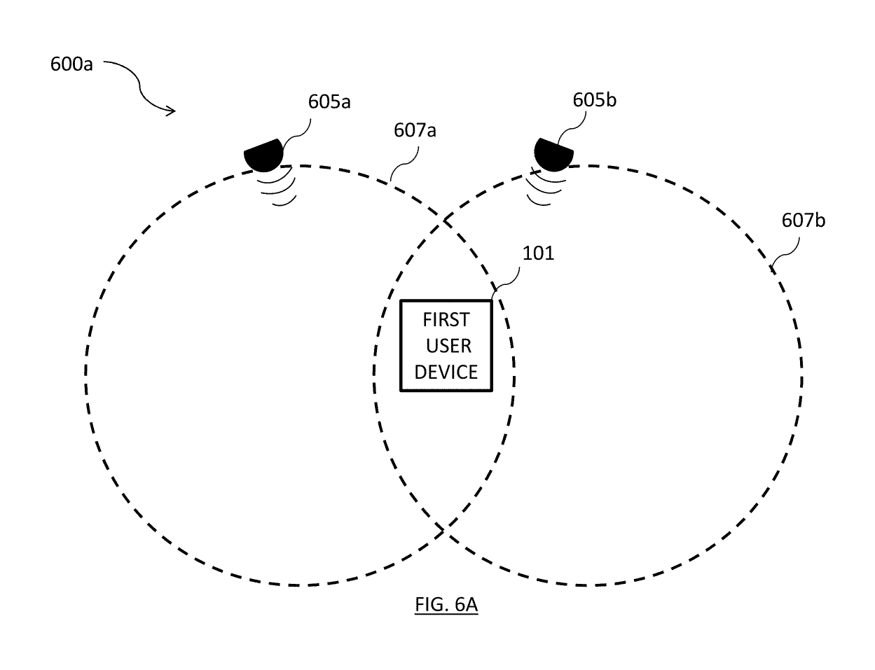

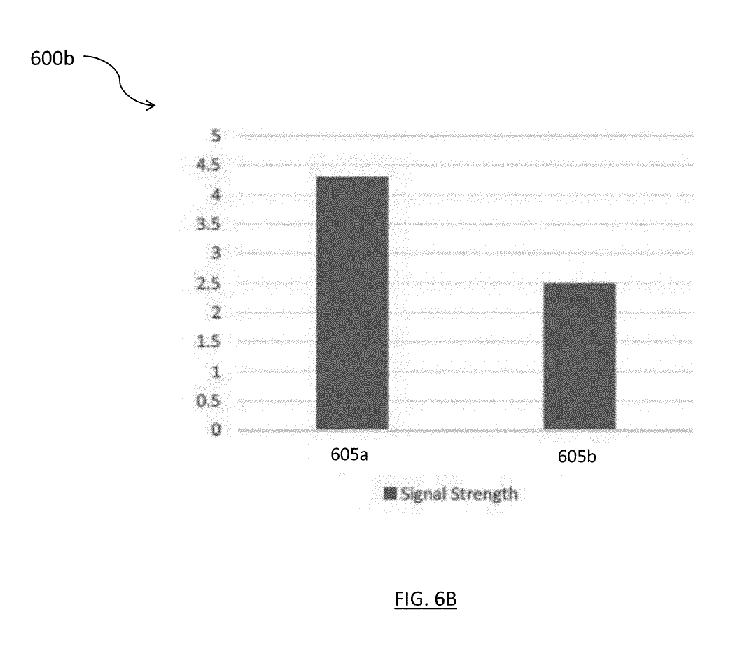

FIGS. 6A and 6B illustrate diagrammatic views of time-stamped location data selection, in accordance with one or more example embodiments. In diagram 600a of FIG. 6A, the user (also the first user device 101 carried by the user) may move from PSD 607a corresponding to communication device 605a to 607b corresponding to communication device 605b. The processor of the first user device 101 may be configured to analyze signal strengths of signals transmitted by each of the communication devices (605a, 605b). When user device 101 moves from the PSD 607a to 607b, the processor of the first user device 101 may compare signal strength of each signal associated with those communication devices (605a, 605b). If signal strength of the signal associated with the communication device 605a is more than that associated with the communication device 605b, the processor may consider the signal associated with the communication device 605a. As shown in bar diagram 600b of FIG. 6B, the signal associated with the communication device 605a may exhibit a signal strength of 4.25 units, while the signal associated with the communication device 600b may exhibit a signal strength of 2.5 units. However, if the signals associated with both the communication devices (605a, 605b) are of same strength, the processor of the first user device 102 may communicate with a historic signal strength database and may retrieve information regarding historic trends of the signals. In this check, the processor may select the signal with progressively increasing signal strength and reject the signal with progressively decreasing signal strength.

FIG. 7 illustrates an exemplary embodiment of a communication diagram, in accordance with an embodiment of the present invention. The communication diagram 700 of FIG. 7 depicts communication among the first user device 101, the electronic device 109 and a second user device 703 over a communication network 705, similar to the communication network 205. The electronic device 109 may actuate connection between the first user device 101 and the second user device 703 based on a trigger event. In some example embodiments, the trigger event may be one of a distance proximity event or a manual actuation event. The connection between the first user device 101 and the second user device 709 may be triggered based on the distance proximity event when the user carrying the first user device 101 comes physically within a threshold distance of the second user device 709. The threshold distance may be set by the administrator. The electronic device 109 may receive location data of the first user device 101 and may determine whether the first user device is within the threshold distance. If the first user device 101 is within the threshold distance, the electronic device 109 may determine identity of each of the first user device 101 and the second user device 703 and may send a notification to each of the user devices (101 and 703) on successful identification of the devices (101 and 703). The electronic device 109 may identify the first user device 101 based on the first user device identification data, as depicted in the method 300 illustrated in FIG. 3. Likewise, identification of the second user device 703 may be based on electronic device authentication data of the second user device 703. Further, the devices (101 and 703) may automatically initiate communication with each other. Once communication between the first user device 101 and the second user device 703 is established, the electronic device 109 may be communicated by both the user devices (101 and 703) regarding the successful communication. The second user device 703 may include a display device configured to display the contextual data. The display device may include a Liquid Crystal Display (LCD), a Light Emitting Diode display (LED), an Organic LED, and Amorphous LED, or a plasma screen In some example embodiments, the second user device 703 may include any of the at least one communication device (105a-105f).

In some example embodiments, the first user device 101 and the second user device 703 may be connected through a communication network different from a communication network through which the first user device 101 and the second user device 703 may be connected to the electronic device 109.

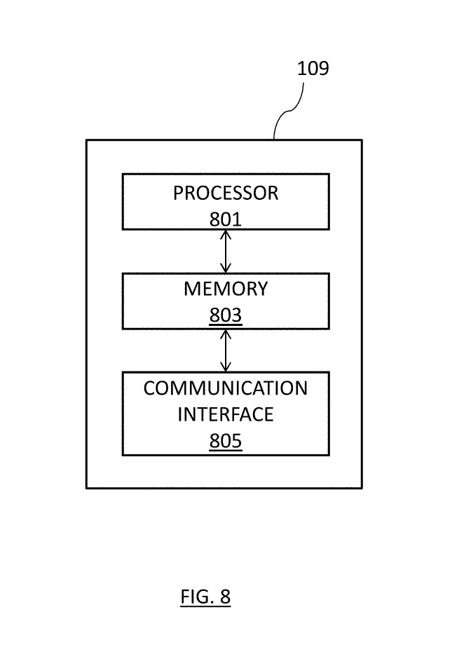

In one or more example embodiments, the first user device 101 may communicate with more devices/systems other than the second user device 703 for transmitting the contextual data, and the electronic device 109 may control transmission of the contextual data to those devices/systems. Additionally or alternatively, in some example embodiments, apart from controlling transmission of contextual data between two or more devices/systems, the electronic device 109 may transmit contextual data (e.g. updated user information) to the first user device 101 or the second user device 703 or any third party devices through a communication interface 805, as depicted in the exemplary embodiment of FIG. 8.

FIG. 8 illustrates block diagram view of the electronic device 109, according to one or more example embodiments. The electronic device 109 may include a processing means such as at least one processor 801, a storage means such as at least one memory 803, and a communication means such as at least one communication interface 805. The processor 801 may retrieve computer program code instructions that may be stored in the memory 803 for execution of the computer program code instructions.

The processor 801 may be embodied in a number of different ways, similar to the processor 401. Additionally or alternatively, the processor 801 may include one or more processors capable of processing large volumes of workloads and operations to provide support for big data analysis. In an example embodiment, the processor 801 may be in communication with a memory 803 via a bus for passing information among components of electronic device 109. The memory 803 may be non-transitory and may include, for example, one or more volatile and/or non-volatile memories, similar to the memory 403. The communication interface 805, similar to the communication interface 407, may provide an interface for accessing various features and data stored in the electronic device 109. In some example embodiments, the processor 801 may be configured to authenticate connection of the first user device 101 with other devices/systems, and notify the first user device 101 and the other devices/systems once the connection is authenticated. Further, the processor 801 may maintain a database within the memory 803 corresponding to each device (e.g. the first user device 101). whose connection with any other device may be authenticated by the processor 801. The processor 801 may retrieve necessary data from the database to determine which device needs to connect to with other devices, as well as whether threshold distance criterion for each device has been met, geolocation coordinates of devices that need to connect to other devices, and the like. The processor 801 may further be configured to determine contextual data for the devices that are connected at a point in time. In some example embodiments, the processor 801 may correlate time-dependent contextual data associated with the devices. For example, contextual data associated with a device at 10:00 am of a day may be different from contextual data associated with the same device at 5:00 pm of the same day. In these embodiments, the memory 803 may store the contextual data determined by the processor 801.

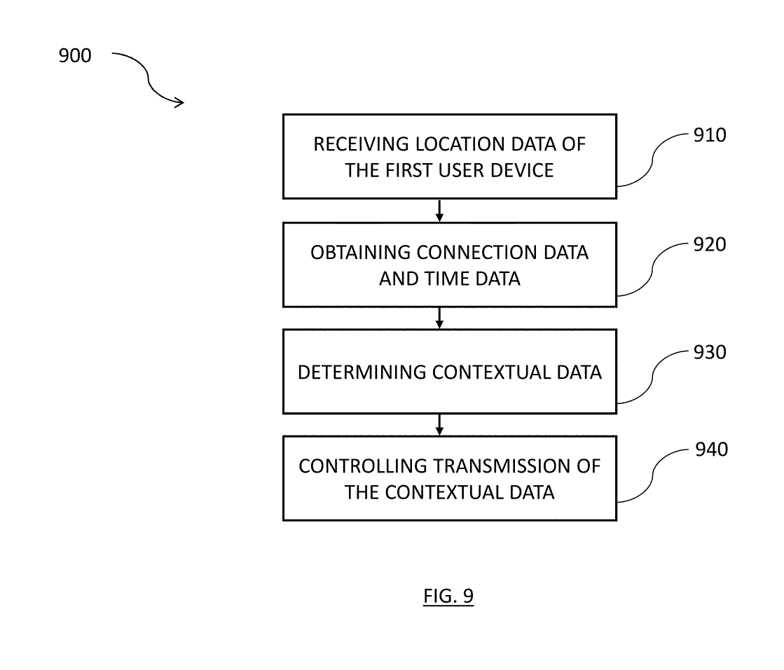

FIG. 9 illustrates a flowchart illustrative of a method according to example embodiments of the present invention. Accordingly, blocks of the flowcharts support combinations of means for performing the specified functions and combinations of operations for performing the specified functions for performing the specified functions. It will also be understood that one or more blocks of the flowcharts, and combinations of blocks in the flowcharts, can be implemented by special purpose hardware-based computer systems which perform the specified functions, or combinations of special purpose hardware and computer instructions.

FIG. 9 illustrates a flowchart depicting steps in a method 900 for controlling transmission of the contextual data, according to an example embodiment of the present invention. At step 910, the method 900 may include receiving location data of the first user device 101. As described in previous embodiments, the first user device 101 may include sensor unit that may capture location data of the first user device 101. The location data may indicate a location of the first user device 101. The method may further include, at step 920, obtaining connection data corresponding to a connection between the first user device 101 and at least one second user device (e.g. 703) and time data associated with the connection data, based on the location data. The method may comprise, at step 930, determining the contextual data corresponding to the first user device 101, based on the time data associated with the connection data. The method may further comprise, at step 940, controlling transmission of the contextual data from the first user device 101 to the at least one second user device, based on the connection data. As mentioned in the previous embodiments, the contextual data may include one or more of personal data of a user of the first user device 101 or electronic medical record data of the user. The at least one second device may comprise a display device configured to display the contextual data.

Additionally, various other steps not shown in FIG. 9 may also be included in the method 900. For example, the method 900 may further comprise actuating connection between the first user device 101 and the at least one electronic device based on a trigger event. The trigger event may be one of a distance proximity event or a manual actuation event. The method 900 may further include receiving first user device identification data of the first user device 101 and authenticating the first user device 101 for connection with the at least one electronic device, based on the first user device identification data and the location data. The step of authenticating the first user device 101 for connection with the at least one electronic device based on the location of the first user device may indicate the first user device 101 being within a threshold distance from the at least one electronic device. The method 900 may further comprise communicating a notification associated with the connection data to each of the first user device 101 and the at least one electronic device.

In an example embodiment, a system or an apparatus (e.g., the electronic device 109) for performing the method of FIG. 9 above may comprise a processor configured to perform some or each of the operations (910-940) described above. The processor may, for example, be configured to perform the operations by performing hardware implemented logical functions, executing stored instructions, or executing algorithms for performing each of the operations. Alternatively, the system may comprise means for performing each of the operations described above. In this regard, according to an example embodiment, examples of means for performing operations 910-940 may comprise, for example, the processor and/or a device or circuit for executing instructions or executing an algorithm for processing information as described above.

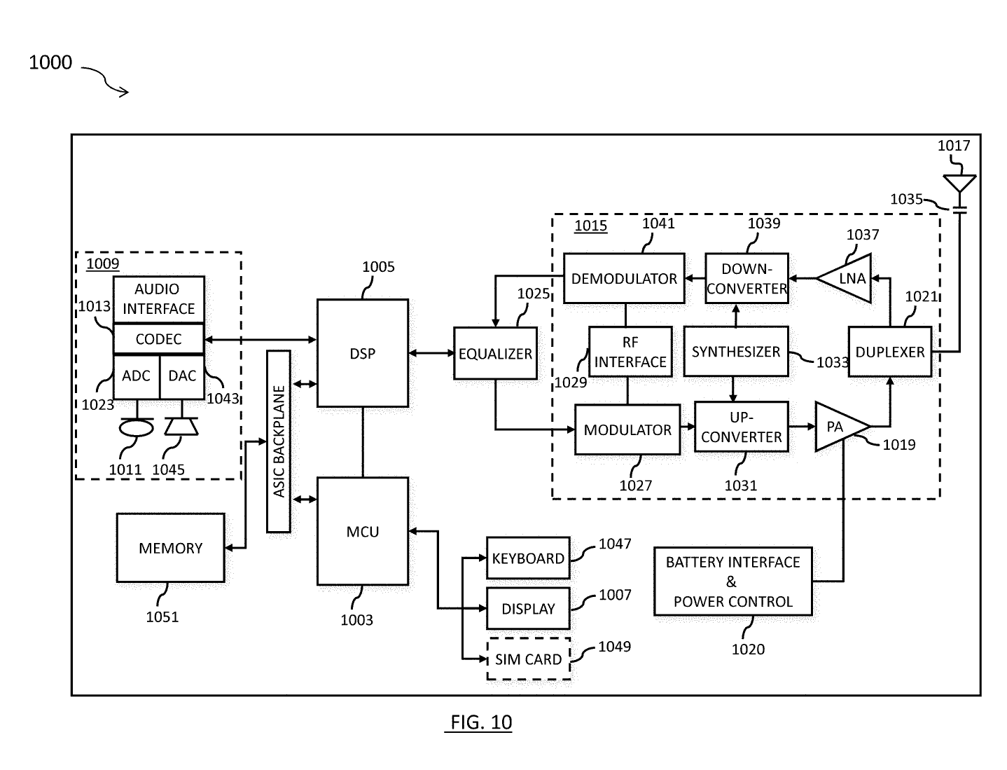

FIG. 10 is a diagram of exemplary components of a mobile station 1000 (e.g., handset or part thereof) capable of operating in the system of FIG. 2, according to one embodiment. Generally, a radio receiver is often defined in terms of front-end and back-end characteristics. The front-end of the receiver encompasses all of the Radio Frequency (RF) circuitry whereas the back-end encompasses all of the base-band processing circuitry. Pertinent internal components of the telephone include a Main Control Unit (MCU) 1003, a Digital Signal Processor (DSP) 1005, and a receiver/transmitter unit including a microphone gain control unit and a speaker gain control unit. A main display unit 1007 provides a display to the user in support of various applications and mobile station functions that offer automatic contact matching. An audio function circuitry 1009 includes a microphone 1011 and microphone amplifier that amplifies the speech signal output from the microphone 1011. The amplified speech signal output from the microphone 1011 is fed to a coder/decoder (CODEC) 1013.

A radio section 1015 amplifies power and converts frequency in order to communicate with a base station, which is included in a mobile communication system, via antenna 1017. The power amplifier (PA) 1019 and the transmitter/modulation circuitry are operationally responsive to the MCU 1003, with an output from the PA 1019 coupled to the duplexer 1021 or circulator or antenna switch, as known in the art. The PA 1019 also couples to a battery interface and power control unit 1020.

In use, a user of mobile station 1001 speaks into the microphone 1011 and his or her voice along with any detected background noise is converted into an analog voltage. The analog voltage is then converted into a digital signal through the Analog to Digital Converter (ADC) 1023. The control unit 1003 routes the digital signal into the DSP 1005 for processing therein, such as speech encoding, channel encoding, encrypting, and interleaving. In one embodiment, the processed voice signals are encoded, by units not separately shown, using a cellular transmission protocol such as global evolution (EDGE), general packet radio service (GPRS), global system for mobile communications (GSM), Internet protocol multimedia subsystem (IMS), universal mobile telecommunications system (UMTS), etc., as well as any other suitable wireless medium, e.g., microwave access (WiMAX), Long Term Evolution (LTE) networks, code division multiple access (CDMA), wireless fidelity (Wi-Fi), satellite, and the like.

The encoded signals are then routed to an equalizer 1025 for compensation of any frequency-dependent impairments that occur during transmission though the air such as phase and amplitude distortion. After equalizing the bit stream, the modulator 1027 combines the signal with a RF signal generated in the RF interface 1029. The modulator 1027 generates a sine wave by way of frequency or phase modulation. In order to prepare the signal for transmission, an up-converter 1031 combines the sine wave output from the modulator 1027 with another sine wave generated by a synthesizer 1033 to achieve the desired frequency of transmission. The signal is then sent through a PA 1019 to increase the signal to an appropriate power level. In practical systems, the PA 1019 acts as a variable gain amplifier whose gain is controlled by the DSP 1005 from information received from a network base station. The signal is then filtered within the duplexer 1021 and optionally sent to an antenna coupler 1035 to match impedances to provide maximum power transfer. Finally, the signal is transmitted via antenna 1017 to a local base station. An automatic gain control (AGC) can be supplied to control the gain of the final stages of the receiver. The signals may be forwarded from there to a remote telephone which may be another cellular telephone, other mobile phone or a land-line connected to a Public Switched Telephone Network (PSTN), or other telephony networks.

Voice signals transmitted to the mobile station 1001 are received via antenna 717 and immediately amplified by a low noise amplifier (LNA) 1037. A down-converter 1039 lowers the carrier frequency while the demodulator 1041 strips away the RF leaving only a digital bit stream. The signal then goes through the equalizer 1025 and is processed by the DSP 705. A Digital to Analog Converter (DAC) 1043 converts the signal and the resulting output is transmitted to the user through the speaker 1045, all under control of a Main Control Unit (MCU) 1003—which can be implemented as a Central Processing Unit (CPU) (not shown).

The MCU 1003 receives various signals including input signals from the keyboard 1047. The keyboard 1047 and/or the MCU 703 in combination with other user input components (e.g., the microphone 1011) comprise a user interface circuitry for managing user input. The MCU 1003 runs a user interface software to facilitate user control of at least some functions of the mobile station 1001 to provide machine learning of physical dividers. The MCU 1003 also delivers a display command and a switch command to the display 1007 and to the speech output switching controller, respectively. Further, the MCU 1003 exchanges information with the DSP 1005 and can access an optionally incorporated SIM card 1049 and a memory 1051. In addition, the MCU 1003 executes various control functions required of the station. The DSP 1005 may, depending upon the implementation, perform any of a variety of conventional digital processing functions on the voice signals. Additionally, DSP 1005 determines the background noise level of the local environment from the signals detected by microphone 1011 and sets the gain of microphone 1011 to a level selected to compensate for the natural tendency of the user of the mobile station 1001.

The CODEC 1013 includes the ADC 1023 and DAC 1043. The memory 1051 stores various data including call incoming tone data and is capable of storing other data including music data received via, e.g., the global Internet. The software module could reside in RAM memory, flash memory, registers, or any other form of writable computer-readable storage medium known in the art including non-transitory computer-readable storage medium. For example, the memory device 1051 may be, but not limited to, a single memory, CD, DVD, ROM, RAM, EEPROM, optical storage, or any other non-volatile or non-transitory storage medium capable of storing digital data.

An optionally incorporated SIM card 1049 carries, for instance, important information, such as the cellular phone number, the carrier supplying service, subscription details, and security information. The SIM card 1049 serves primarily to identify the mobile station 1001 on a radio network. The card 1049 also contains a memory for storing a personal telephone number registry, text messages, and user specific mobile station settings.

In this way, example embodiments of the present invention result in update of time-stamped, synchronized information regarding movements of a user through different areas of a facility, such as a healthcare facility, thus aid in providing new information to the healthcare facility regarding movements of the user within premises of the healthcare facility. The present invention provides information about precise location of the user in healthcare facility premises. For example, the present invention enables monitoring of movements of a psychiatric patient in a hospital. Thus, the present invention aids in tracking the psychiatric patient in the hospital and provides assistance to authority of the hospital to ensure that the psychiatric patient is in authorized room. Moreover, the present invention may aid in immediately locating an elderly person fallen in some corner of the hospital and admitting the elderly person in emergency ward in time, thus saving a life. Whenever the user putting on a wearable device (e.g. the first user device 101) visits a healthcare centre, the wearable device may be connected wirelessly to a smart device (having display provision) of the healthcare centre and medical information related to the user may be shared with the user, doctors and health insurance providers. In case the healthcare facility recommends any diagnostic test according to schedule of the user based upon contextual data captured as per the user's schedule, the electronic device may send contextual data to a laboratory for communicating with the user for the prescribed test. In this way, the contextual data in various nodes of a network may benefit several human parties in that network, who may require that contextual data to perform specific tasks or decide on additional ones.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

What is claimed is:

1. A system for updating user information, comprising:
a first device comprising:
first circuitry configured to:
generate motion data of the first device;
receive from at least one communication device, time-stamped location data of the first device, based on the generated motion data;
generate time synchronized user data based on the time-stamped location data and device data of the first device; and
transmit the time synchronized user data; and
a second device communicably coupled to the first device, comprising:
second circuitry configured to:
receive the time synchronized user data; and
update the user information based on the time synchronized user data.

2. The system of claim 1, wherein the time-stamped location data comprises geolocation data of the first device and timestamp data associated with the geolocation data.

3. The system of claim 1, wherein the device data comprises device identifier data of the first device and profile data of a user associated with the first user device.

4. The system of claim 3, wherein the profile data of the user comprises one or more of name, age, sex, health insurance number, address, medical history of the user.

5. The system of claim 1,
wherein the second circuitry is further configured to store the user information, and
wherein the user information corresponds to an electronic medical record of a user of the first device.

6. The system of claim 1, wherein the time synchronized user data comprises geolocation data of the first device, timestamp data associated with the geolocation data, device identifier data of the first device, and profile data of a user of the first device.

7. The system of claim 1, wherein the first device is a wearable device.

8. The system of claim 1, wherein the communication device is an RFID transponder.

9. The system of claim 1, wherein the second device is a database server.

10. A portable device, comprising:
circuitry configured to:
generate motion data of the portable device;
receive from at least one communication device, time-stamped location data of the portable device, based on the generated motion data;
generate time synchronized user data based on the time-stamped location data and device data of the portable device; and
transmit the time synchronized user data to a server device for update of user information.

11. The portable device of claim 10, wherein the time-stamped location data comprises geolocation data of the portable device and timestamp data associated with the geolocation data.

12. The portable device of claim 10, wherein the device data comprises device identifier data of the portable device and profile data of a user associated with the portable device.

13. The portable device of claim 12, wherein the profile data of the user comprises one or more of name, age, sex, health insurance number, address, medical history of the user.

14. The portable device of claim 10, wherein the user information corresponds to an electronic medical record of a user associated with the portable device.

15. The portable device of claim 10, wherein the time synchronized user data comprises geolocation data of the portable device, timestamp data associated with the geolocation data, device identifier data of the portable device, and profile data of a user of the portable device.

16. A method for updating user information, comprising:
generating by a first device, motion data of the first device;
receiving by the first device, time-stamped location data of the first device based on the generated motion data, from at least one communication device;
generating by the first device, time synchronized user data, based on the time-stamped location data and device data of the first device; and
updating by a second device, the user information based on the time synchronized user data.

17. The method of claim 16, wherein the time-stamped location data comprises geolocation data of the first device and timestamp data associated with the geolocation data.

18. The method of claim 16, wherein the device data comprises device identifier data of the first device and profile data of a user associated with the first user device.

19. The method of claim 18, wherein the profile data of the user comprises one or more of name, age, sex, health insurance number, address, medical history of the user.

20. The method of claim 16, wherein the user information corresponds to an electronic medical record of a user of the first device.
